(19) United States
(12) Patent Application Publication
(10) Pub. No.: US 2024/0225631 A1
(43) Pub. Date: Jul. 11, 2024

(54) Labral Anchor System and Method

(71) Applicant: AEVUMED, INC., Malvern, PA (US)

(72) Inventors: Miles O. Curtis, Philadelphia, PA (US); Saif Khalil, Malvern, PA (US)

(21) Appl. No.: 18/405,123

(22) Filed: Jan. 5, 2024

Related U.S. Application Data
(60) Provisional application No. 63/478,577, filed on Jan. 5, 2023.

(51) Int. Cl. A61B 17/04; A61B 17/00

(52) U.S. Cl. CPC A61B 17/0401; A61B 17/0482; A61B 2017/00469; A61B 2017/0409; A61B 2017/0414; A61B 2017/0438

(57) ABSTRACT

A suture anchor system includes an anchor having an elongate cylindrical body extending between a proximal end and a distal end, and an eyelet traversing a portion of the elongate cylindrical body, a drill guide having an interior chamber in fluid communication with a distal opening, an elongate anchor inserter having a distal surface configured to interface with a proximal surface of the anchor, and an elongate drill member having a distal drilling tip. The interior chamber is configured to at least partially house the elongate anchor inserter and the elongate drill member.

### CROSS-REFERENCE TO RELATED APPLICATIONS

This application claims priority to U.S. provisional application No. 63/478,577, filed Jan. 5, 2023 incorporated herein by reference in its entirety.

### BACKGROUND OF THE INVENTION

The labrum is a cup-shaped rim of cartilage that lines and reinforces the ball-and-socket joint of the shoulder. The labrum is the attachment site for the shoulder ligaments and supports the ball-and-socket joint as well as the rotator cuff tendons and muscles. It contributes to shoulder stability and, when torn, can lead to partial or complete shoulder dislocation and may require surgical repair.

Suture anchors are commonly employed in surgical procedures to attach soft tissue such as tendons or ligaments to bone. For instance, in a rotator cuff repair, suture is passed through a detached or damaged portion of a rotator cuff tendon. A suture anchor is implanted into the adjacent bone. By attaching the suture to the anchor, the tendon is pulled into contact with the bone to promote adhesion of the tendon to the bone. Such procedures are often performed arthroscopically through a narrow cannula. This reduces trauma to the patient but makes attachment of the suture to the anchor using a knot more difficult.

Labral anchors may be employed which allow a surgeon to affix soft tissue without having to tie a knot. Existing labral anchors pre-tension a suture to a desired degree prior to securing the suture by trapping it between outer threading of the labral suture anchor and adjacent bone. As the anchor is screwed in, the tension in the suture may be loosened or overtightened with no means to re-tension the suture other than removing the anchor. The suture may also interfere with the engagement between the anchor ribs and adjacent bone, leading to poor fit, compromised healing or resorption, and possibly anchor or suture pullout.

Thus, there is a need in the art for improved labral anchors allowing for sutures to be re-tensioned to enhance labral repair and healing by securing sutures without knots or trapping the sutures against bone. The present invention meets this need.

### SUMMARY OF THE INVENTION

In one embodiment, a suture anchor system includes an anchor having an elongate cylindrical body extending between a proximal end and a distal end, and an eyelet traversing a portion of the elongate cylindrical body; a drill guide having an interior chamber in fluid communication with a distal opening; an elongate anchor inserter having a distal surface configured to interface with a proximal surface of the anchor; and an elongate drill member having a distal drilling tip; wherein the interior chamber is configured to at least partially house the elongate anchor inserter and the elongate drill member. In one embodiment, the distal opening is configured to accept advancement therethrough of the distal drilling tip while the anchor and elongate anchor inserter remain housed in the lumen. In one embodiment, a surface of the interior chamber comprises a ramp structure that tapers towards the distal opening. In one embodiment, the ramp structure terminates at a distal chamber portion which has a smaller diameter than portions of the interior chamber proximal to the ramp structure. In one embodiment, the interior chamber is configured to arrest movement of the anchor and elongate anchor inserter while the distal drilling tip is advanced through the distal opening. In one embodiment, the interior chamber is configured to allow advancement of the anchor and elongate anchor inserter through the distal opening after the elongate drill member is retracted from the distal opening. In one embodiment, the elongate anchor inserter and the elongate drill member are housed in parallel within the interior chamber. In one embodiment, the interior chamber comprises a single lumen configured to house both the elongate anchor inserter and the elongate drill member. In one embodiment, the interior chamber comprises a first lumen configured to house the elongate anchor inserter and a second lumen configured to house the elongate drill member, wherein the first and second lumen are in fluid communication with the distal opening. In one embodiment, a suture guide slot configured in a wall of the interior chamber and terminating distally at the distal opening. In one embodiment, the wall comprises a first and second portion of the interior chamber, the second portion of the interior chamber configured distal of the first portion of the interior chamber, and the second portion of the interior chamber having a smaller diameter than a first portion of the interior chamber. In one embodiment, the suture guide slot comprises at least one curved portion. In one embodiment, the curved portion is disposed adjacent to a surface of the interior chamber comprising a ramp structure that tapers towards the distal opening. In one embodiment, the suture guide slot comprises at least one straight portion. In one embodiment, the suture guide comprises a proximal portion that tapers down to a narrower distal portion. In one embodiment, the distal surface of the elongate anchor inserter comprises a protrusion configured to mate with the anchor. In one embodiment, the proximal surface of the anchor comprises a protrusion configured to mate with the elongate anchor inserter. In one embodiment, the protrusion configured to mate with the elongate anchor inserter is configured to insert into a distal opening of the elongate anchor inserter. In one embodiment, the anchor comprises a proximal opening traversing the elongate cylindrical body configured proximal of the eyelet. In one embodiment, a kit includes the suture anchor system a suture preloaded within the proximal opening; and the anchor preloaded onto the elongate anchor inserter

In some aspects, a suture anchor system, having an anchor having an elongate cylindrical body extending between a proximal end and distal end, an elongate driver opening extending distally though the body from a proximal end opening, and a suture eyelet traversing the body and the elongate driver opening, tapering proximally into a pinch slit having a pinch slit opening, and a driver insertion tool having a pair of distal tines separated by a gap, wherein the pinch slit opening is unobstructed when the pair of distal tines are fully inserted into the elongate driver opening.

In some embodiments, the eyelet is formed from a lumen passing through the elongate cylindrical body from a first lateral opening to an opposing second lateral opening. In some embodiments, the elongate cylindrical body has a substantially oblong eyelet with a narrower proximal pinch slit configured to secure one or more lengths of suture threads. In some embodiments, the elongate cylindrical body has an opposing longitudinal suture recesses configured to receive one or more lengths of suture thread.

In some embodiments, the elongate cylindrical body has a ribbed exterior configured for push-in insertion. In some embodiments, the elongate cylindrical body has an exterior having a plurality of ribs configured for push-in insertion, wherein each rib extends out of the body and is angled outwards towards the proximal end of the anchor.

In some embodiments, the anchor has a distal piercing tip having a shape selected from the group consisting of: a conical tip, a pyramidal tip, a hypodermic needle tip, a wedge tip, a bladed tip, and a trocar tip. In some embodiments, the suture anchor system is fabricated from a material selected from the group consisting of: PEEK, PAEK, PEKK, titanium, titanium-alloys, bioabsorbables, platinum, plastic, metal, and combinations thereof.

In some embodiments, the anchor body has a length between about 5 mm to about 50 mm and a diameter between about 5 mm to about 20 mm. In some embodiments, the anchor is configured to engage with the pair of distal tines.

In some embodiments, the tines extend into the anchor body and obstruct suture passing through the eyelet. In some embodiments, the tines extend into the anchor body and do not obstruct suture passing through the eyelet. In some embodiments, the driver is made from a metal and the anchor body is metal-free.

In some aspects, a method of attaching soft tissue to bone, the method having the steps of providing a labral suture anchor having an elongate cylindrical body with proximal and distal ends, opposing longitudinal suture reliefs, and a longitudinal suture eyelet with oblong cross-sectional shape and a proximal pinch slit, passing a suture through the eyelet, passing the suture around a soft tissue, pre-tensioning the suture to a first tension, driving the suture anchor into a bone of a subject, tensioning the suture to a second tension.

In some embodiments, the labral suture anchor is driven into a preformed hole in a bone of a subject. In some embodiments, the hole is formed using a tool selected from the group consisting of: an awl, a tap, and a drill. In some embodiments, the labral suture anchor is punched or hammered directly into a bone of a subject without a preformed hole.

In some embodiments, the at least one suture thread is re-tensioned. In some embodiments, a remainder of each of the at least one suture thread is positioned in lateral spaces of the suture reliefs adjacent to the anchor body.

### BRIEF DESCRIPTION OF THE DRAWINGS

The following detailed description of exemplary embodiments of the invention will be better understood when read in conjunction with the appended drawings. It should be understood, however, that the invention is not limited to the precise arrangements and instrumentalities of the embodiments shown in the drawings.

FIG. 1A and FIG. 1B depict perspective views of an exemplary labral anchor system having an anchor device and an anchor driver tool. FIG. 1A depicts a perspective view of an exemplary anchor device separated from an exemplary anchor driver. FIG. 1B depicts a perspective view of an exemplary anchor driver engaged with an exemplary anchor device.

FIG. 2 depicts a side view (Left) and a top-down view (Right) of an exemplary labral anchor device.

FIG. 3A and FIG. 3B depict a side view (Left) and perspective view (Right) of a suture loaded in an exemplary anchor system.

FIG. 4A and FIG. 4B depict a series of images illustrating an exemplary method of repairing soft tissue to bone.

FIG. 5A and FIG. 5B depict a series of images illustrating an exemplary method of repairing soft tissue to bone.

FIG. 6A depicts an exemplary anchor system according to aspects of the present invention.

FIG. 6B depicts an exemplary anchor system according to aspects of the present invention.

FIG. 7 depicts an exemplary method of repairing soft tissue to bone using an exemplary anchor system according to aspects of the present invention

FIG. 8A depicts an exemplary method of repairing soft tissue to bone using an exemplary anchor system according to aspects of the present invention.

FIG. 8B depicts an exemplary method of repairing soft tissue to bone using an exemplary anchor system according to aspects of the present invention.

FIG. 9 depicts an exemplary anchor system according to aspects of the present invention.

FIG. 10 depicts an exemplary method of repairing soft tissue to bone using an exemplary anchor system according to aspects of the present invention.

FIG. 11 depicts an exemplary method of repairing soft tissue to bone using an exemplary anchor system according to aspects of the present invention.

FIG. 12A and FIG. 12B depicts an exemplary method of repairing soft tissue to bone using an exemplary anchor system according to aspects of the present invention.

FIG. 13 is a flowchart of an exemplary method of repairing soft tissue to bone.

FIG. 14 is a flowchart of an exemplary method of repairing soft tissue to bone.

FIG. 15 is a flowchart of an exemplary method of repairing soft tissue to bone.

### DETAILED DESCRIPTION

The present invention provides suture anchor systems and devices for attaching soft tissue to bone. The systems and devices include suture anchors configured to engage and fasten sutures using an eyelet with pinch-slit such that the locked sutures are free from frictional engagement with bone, anchor body and anchor ribs.

### Definitions

It is to be understood that the figures and descriptions of the present invention have been simplified to illustrate elements that are relevant for a clear understanding of the present invention, while eliminating, for the purpose of clarity, many other elements typically found in the art. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined elsewhere, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of +20%, +10%, +5%, +1%, and +0.1% from the specified value, as such variations are appropriate.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6, and any whole and partial increments there between. This applies regardless of the breadth of the range.

### Labral Anchor System

In some aspects, the present invention relates to various labral anchor systems comprising a suture anchor and an anchor driver tool. In some examples, the suture anchor is a push-in style anchor having a suture eyelet with pinch slit for receiving and securing at least one suture, and a proximal end opening for receiving an anchor driver, wherein the anchor driver aids in the insertion of the push in style anchor. In some embodiments, the labral anchor system may further comprise an insertion tool and a drill.

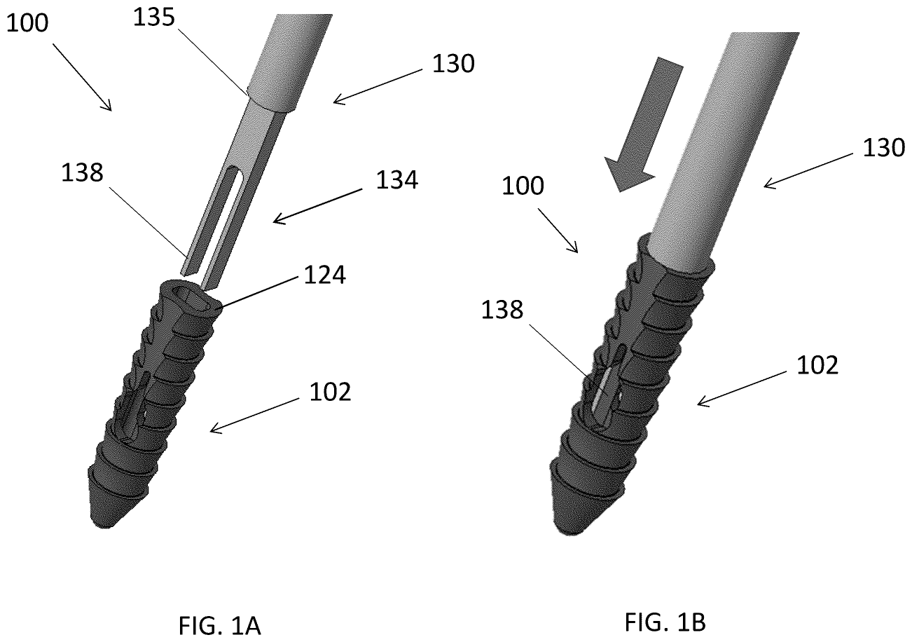

Now referring to FIG. 1A, shown is an exemplary labral anchor system 100 comprising anchor 102 and anchor driver 130. FIG. 1A depicts an exemplary anchor 102 separated from an exemplary anchor driver 130. FIG. 1B depicts anchor driver 130 engaged with anchor 102. A directional arrow indicates the direction along which anchor driver 130 moves to engage with anchor 102.

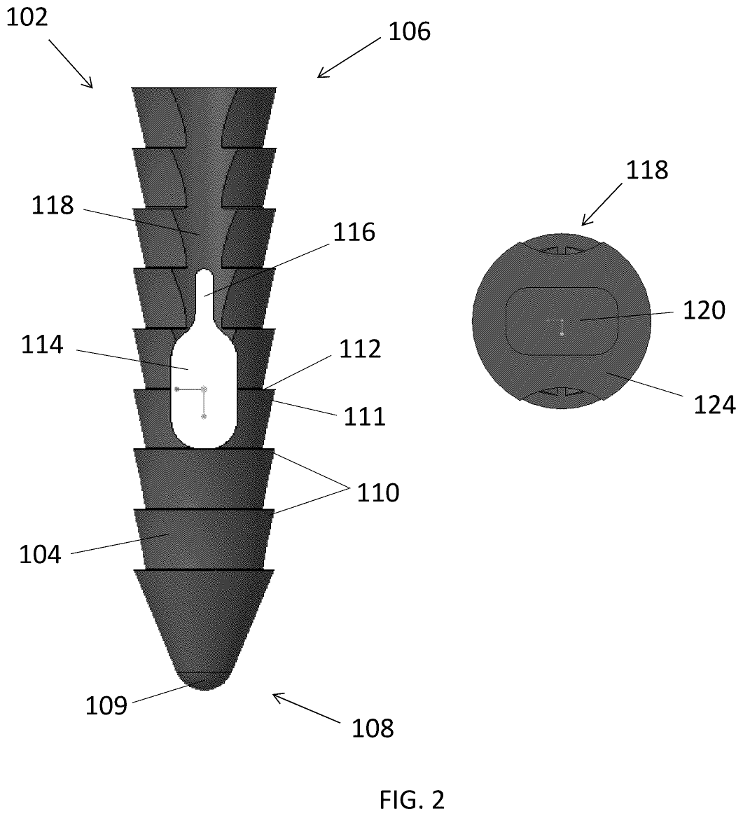

Referring now to FIG. 2, a labral anchor is now described in detail. In some embodiments, anchor 102 has an elongate cylindrical body 104 extending between a proximal end 106 and a distal end 108. In some embodiments, anchor 102 has a plurality of ribs 110 arranged along body 104. In some embodiments, body 104 tapers on the distal end to a distal piercing tip 109. In some embodiments, anchor 102 comprises a longitudinal suture eyelet 114 with a proximal pinch slit 116 extending through body 104 for receiving and securing suture. In some embodiments, anchor 102 comprises at least one suture recess 118 along a portion of body 104 wherein suture may pass through. In some embodiments, anchor 102 comprises proximal end opening 120, for receiving and engaging anchor driver 130 to aid in the insertion of the anchor to bone. In some embodiments, anchor 102 has an elongate driver opening extending distally though body 104 from proximal end opening 120. In some embodiments, anchor 102 comprises proximal face 124 aligned substantially orthogonal to a longitudinal axis of anchor 102.

In some aspects, the present invention relates to a plurality of ribs for a push in style anchor. Again referring to FIG. 2, anchor 102 with plurality of ribs 110 is now described in detail. Each rib of plurality of ribs 110 encircles an exterior of anchor 102 to form a ring-like shape. Plurality of ribs 110 cover at least a portion of an exterior of body 104 of anchor 102. In some embodiments, plurality of ribs 110 cover a length of anchor 102. Each rib of plurality of ribs 110 comprises a rib distal angled face 111 that facilitates insertion in a distal direction towards piercing tip 109, and a rib proximal face 112 angled to resist being pulled out. In some embodiments, rib proximal face 112 is substantially flat and aligned substantially orthogonal to a longitudinal axis of anchor 102. In some embodiments, rib proximal face 112 is angled such that an edge of each rib 110 formed between rib distal angled face 111 and rib proximal face 112 points in a proximal direction. In various embodiments, ribs 110 are machined from the same material as anchor 102.

In some aspects, the present invention relates to a piercing tip of a push in style anchor. In some embodiments, suture anchor 102 comprises a tapered distal end terminating in piercing tip 109. Piercing tip 109 is configured to pierce through and into tissue, including soft tissue and bone tissue. Piercing tip 109 can have any suitable shape, including but not limited to a conical tip, a pyramidal tip, a hypodermic needle tip, a wedge tip, a bladed tip, and the like. In some embodiments, piercing tip 109 is shaped from flat, angled facets. In some embodiments, piercing tip 109 is a trocar tip.

In some aspects, the present invention relates to a suture eyelet of a push in style anchor. In some embodiments, anchor 102 comprises longitudinal suture eyelet 114 proximal to piercing tip 109 to facilitate the receiving, sliding, securing and fixation of pre-loaded and/or knotless suture. In some embodiments, anchor 102 comprises suture eyelet 114 extending through body 104 creating a lumen wherein suture may pass through. In some embodiments, suture eyelet 114 traverses the body of anchor 102 and the elongate driver opening. Suture eyelet 114 is configured to receive suture threads and is formed by a lumen passing through the body 104 of anchor 102 from a first lateral opening to an opposing second lateral opening. Suture eyelet 114 can have any suitable cross-sectional shape, including oblong, circular, elliptical, rectangular, and the like. In some embodiments, suture eyelet 114 is sized to freely pass a plurality of suture threads as well as any knots formed in the suture threads. In some embodiments, suture eyelet 114 comprises a substantially oblong cross-sectional shape with a narrower proximal pinch slit 116. In some embodiments, suture eyelet 114 tapers proximally into pinch slit 116 having a pinch slit opening. In some embodiments, the suture eyelet 114 may be preloaded with one or more sutures.

In some embodiments, anchor 102 comprises a narrow pinch slit 116 on the proximal end of suture eyelet 114, wherein the suture may be secured. In some embodiments, suture eyelet 114 holds suture locked in at least one position. In some embodiments, pinch slit 116 offers a high-friction slide wherein the suture may be hand-tensioned but cannot be moved by the movement of the attached bone or tissue. In some embodiments, pinch slit 116 has ramped features that allow for the suture to only be tightened in one direction.

In some aspects, the present invention relates to at least one suture recess 118 on anchor 102. Again referring to FIG. 2, suture recess 118 extends along a portion of body 104 and comprises at least one cut out region in body 104. In some embodiments, suture recess 118 is a relief in body 104 from the proximal end of eyelet 114 to proximal face 124. In one example, anchor 102 has recess 118 on laterally opposed sides aligning with the openings of suture eyelet 114, and extending to the proximal end of anchor 102. In some embodiments, suture may pass unrestricted from the proximal end of anchor 102, along a first suture recess 118, through eyelet 114 and along a second suture recess 118.

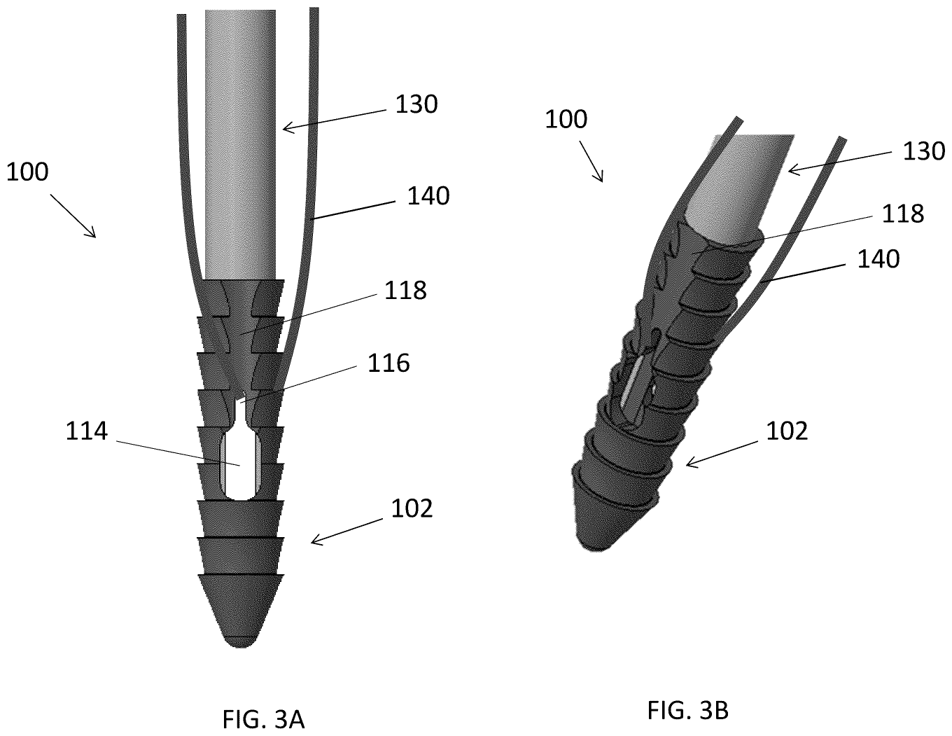

Now referring to FIG. 3A and FIG. 3B, shown is suture 140 loaded into an exemplary anchor 102 according to aspects of the present invention. In some embodiments, the lateral spaces formed by a first and second suture recess form channels through suture 140 may pass through without frictional engagement with anchor 102 or the bone. In some embodiments, the suture may pass through suture eyelet 114 and suture recess 118 alongside the body of anchor 102 and the surrounding bone. In some embodiments, suture recess 118 is a non-captivating feature that allows the suture to freely move vertically in the recess to be tensioned or loosened.

Aspects of the present invention relate to a mating interface and/or engagement structure for the driver and anchor according to aspects of the present invention. Anchor 102 may comprise one or more engagement structures compatible with a distal end of anchor driver 130. For example, as depicted in FIG. 2, anchor 102 has proximal end opening 120 with an elongate channel sized to match tines 138 of a driver 130, wherein the engagement between driver 130 and anchor 102 can be a friction fit.

In some embodiments, anchor 102 has proximal end opening 120 for receiving an anchor driver. In some embodiments, proximal end opening 120 is a driver channel positioned on proximal face 124 to accept the insertion of a driver. Now referring again to FIG. 1, in some embodiments, tines 138 of driver 130 contacts the elongate channel of proximal end opening 120 to form a friction fit wherein the anchor may be inserted and/or rotated into bone. In some embodiments, proximal end opening 120 is square in shape. In some embodiments, proximal end opening 120 is rectangular in shape. In some embodiments, proximal end opening 120 is polygonal in shape. In some embodiments, proximal end opening 120 is star shaped. In some embodiments, when driver 130 is fully inserted into anchor 102, distal face 135 of driver 130 contacts proximal face 124 of anchor 102. In some embodiments, distal face 135 of driver 130 prevents tines 138 from extending too far into end opening 120.

In some aspects, the present invention relates to a driver used for inserting an anchor into bone. In some embodiments, driver 130 comprises proximal end 132 and distal end 134 and a length therebetween. In some embodiments, driver 130 comprises distal face 135 along the length of driver 130, proximal to distal end 134. In some embodiments, driver 130 comprises proximal handle 136 used for gripping the driver. In some embodiments, distal end 134 is square in shape. In some embodiments, distal end 134 is rectangular in shape. In some embodiments, distal end 134 is circular in shape. In some embodiments, distal end 134 is polygonal in shape. In some embodiments, distal end 134 is star shaped.

Driver 130 is designed to engage with anchor 102. In some embodiments, driver 130 is used to insert anchor 102 into bone. In some embodiments, driver 130 comprises distal tines 138 used to engage anchor 102. In some embodiments, a pair of tines 138 are separated by a gap. In some embodiments, tines 138 comprise at least one rectangular extrusion on distal end 134. In some embodiments, distal face 135 of driver 130 contacts proximal face 124 of anchor 102 when the anchor is fully inserted on the distal end of the driver. In some embodiments, tines 138 of driver 130 are rectangular in shape, and mate with the elongate channel of proximal end opening 120, wherein the elongate channel is also rectangular in shape, and sized to match tines 138. In some embodiments, distal face 135 of driver 130 prevents tines 138 from extending too far into end opening 120. In some embodiments, the pinch slit opening is unobstructed when the pair of distal tines are fully inserted into the elongate driver opening of anchor 102. In some embodiments, the pinch slit opening is partially obstructed when the pair of distal tines are fully inserted into the elongate driver opening of anchor 102.

In some embodiments, a portion of tines 138 may be inserted into end opening 120 and extend into suture eyelet 114. In some embodiments, tines 138 obstruct suture passing through eyelet 114 and/or pinch slit 116. In some embodiments, tines 138 do not obstruct suture passing through eyelet 114 and/or pinch slit 116.

In some embodiments, engagement between suture anchor 102 and driver 130 can optionally further include a suture or wire attached to suture anchor 102 and used to pulled anchor 102 taut against driver 130.

In one aspect, the present invention relates to qualities for an anchor driver. In some embodiments, driver 130 comprises superelastic, pseudoelastic and/or “shape memory” material, such as nitinol, to allow the driver to advance the anchor along a curved path.

In various embodiments, driver 130 is amenable to any feature or modification to enhance or augment its function. For example, in some embodiments an exterior surface of handle 136 can comprise one or more textured surfaces or ridges configured to enhance a user's grip and to facilitate driving driver 130. In some embodiments, handle 136 can comprise one or more suture holders for temporary cinching or wrapping of suture threads and suture ends. In some embodiments, the proximal end of handle 102 can comprise a high-density polymer or a metal material configured to withstand repeated strikes from a hammer or mallet with minimal or no deformation.

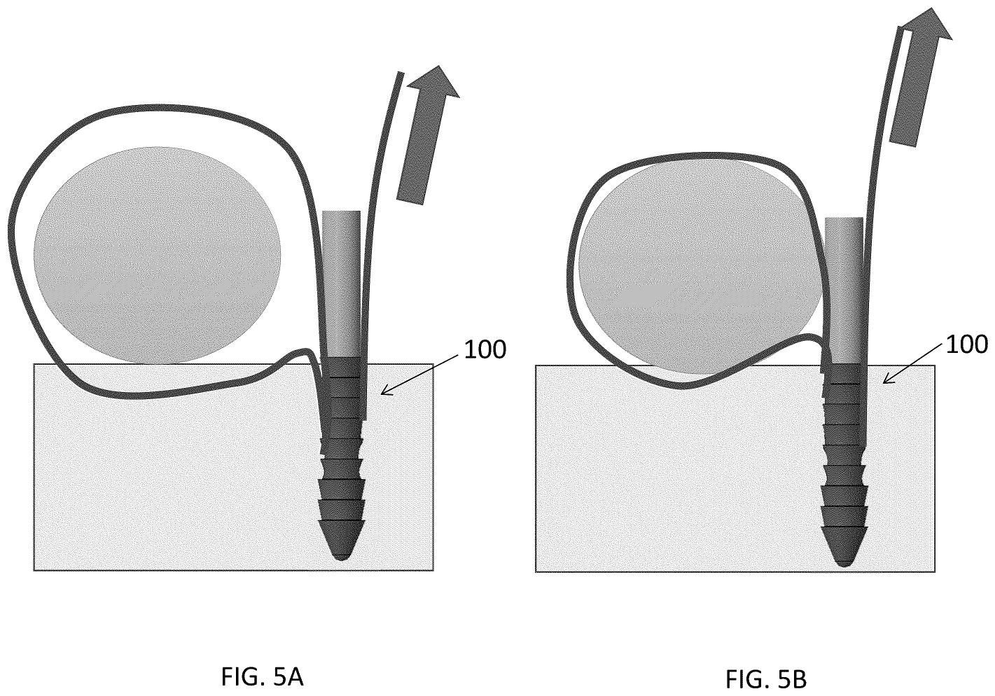

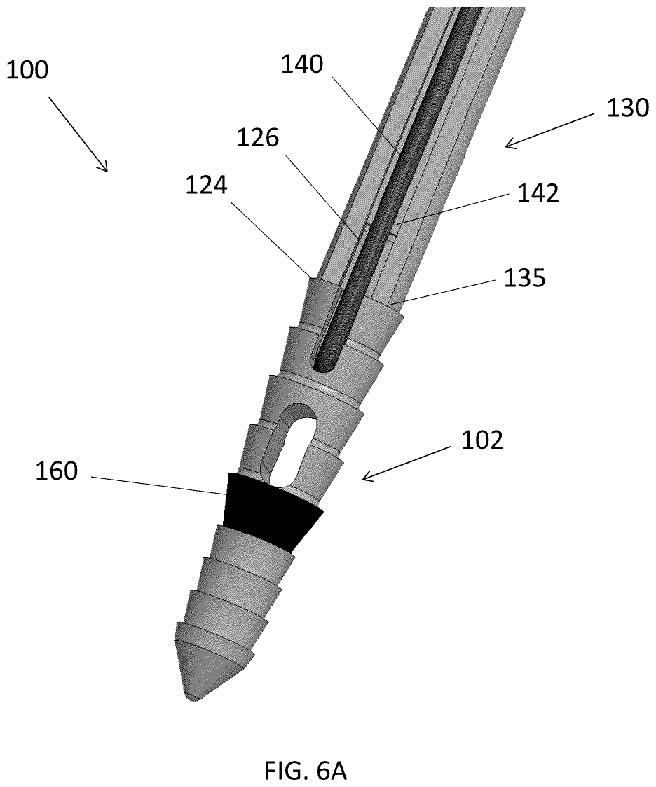

Now referring to FIG. 6A, shown is an exemplary labral anchor system 100 comprising anchor 102 and driver 130. In certain examples, a pre-loaded suture is used to hold anchor to the driver, and following insertion, can be used to repair the labrum. In certain examples, the pre-loaded suture passes through a suture hole in the body of the anchor. In certain examples, the anchor has a laser line to help with the positioning of the anchor.

Referring now to FIG. 6B, an exemplary labral anchor 102 is now described in detail. In some embodiments, anchor 102 has an elongate cylindrical body 104 extending between a proximal end 106 and a distal end 108. In some embodiments, anchor 102 has a plurality of ribs 110 arranged along body 104. In some embodiments, body 104 tapers on the distal end to a distal piercing tip 109. In some embodiments, anchor 102 comprises a longitudinal suture eyelet 114 extending through body 104 for receiving and securing suture. In some embodiments, anchor 102 has a proximal knotless anchor hole 128 for receiving a pre-loaded suture. In some embodiments, anchor 102 comprises at least one suture recess 118 along a portion of body 104 wherein suture may pass through. In some embodiments, anchor 102 comprises proximal end post 126, for engaging anchor driver 130 to aid in the insertion of the anchor to bone. In some embodiments, anchor 102 comprises proximal face 124 aligned substantially orthogonal to a longitudinal axis of anchor 102. In some embodiments, anchor 102 further comprises a proximal end post 126 for engaging driver 130 to help in the insertion of the anchor into bone.

In some aspects, the present invention relates to a plurality of ribs for a push in style anchor. Again referring to FIG. 6B, anchor 102 with plurality of ribs 110 is now described in detail. Each rib of plurality of ribs 110 encircles an exterior of anchor 102 to form a ring-like shape. Plurality of ribs 110 cover at least a portion of an exterior of body 104 of anchor 102. In some embodiments, plurality of ribs 110 cover a length of anchor 102. Each rib of plurality of ribs 110 comprises a rib distal angled face 111 that facilitates insertion in a distal direction towards piercing tip 109, and a rib proximal face 112 angled to resist being pulled out. In some embodiments, rib proximal face 112 is substantially flat and aligned substantially orthogonal to a longitudinal axis of anchor 102. In some embodiments, rib proximal face 112 is angled such that an edge of each rib 110 formed between rib distal angled face 111 and rib proximal face 112 points in a proximal direction. In various embodiments, ribs 110 are machined from the same material as anchor 102. In some embodiments, anchor 102 has narrow distal ribs and wide proximal ribs.

In some aspects, the present invention relates to a piercing tip of a push in style anchor. In some embodiments, suture anchor 102 comprises a tapered distal end terminating in piercing tip 109. Piercing tip 109 is configured to pierce through and into tissue, including soft tissue and bone tissue. Piercing tip 109 can have any suitable shape, including but not limited to a conical tip, a pyramidal tip, a hypodermic needle tip, a wedge tip, a bladed tip, and the like. In some embodiments, piercing tip 109 is shaped from flat, angled facets. In some embodiments, piercing tip 109 is a trocar tip.

In some aspects, the present invention relates to a suture eyelet of a push in style anchor. In some embodiments, anchor 102 comprises longitudinal suture eyelet 114 proximal to piercing tip 109 to facilitate the receiving, sliding, securing and fixation of pre-loaded and/or knotless suture. In some embodiments, anchor 102 comprises suture eyelet 114 extending through body 104 creating a lumen wherein suture may pass through. In some embodiments, suture eyelet 114 traverses the body of anchor 102 and the elongate driver opening. Suture eyelet 114 is configured to receive suture threads and is formed by a lumen passing through the body 104 of anchor 102 from a first lateral opening to an opposing second lateral opening. Suture eyelet 114 can have any suitable cross-sectional shape, including oblong, circular, elliptical, rectangular, and the like. In some embodiments, suture eyelet 114 is sized to freely pass a plurality of suture threads as well as any knots formed in the suture threads. In some embodiments, suture eyelet 114 comprises a substantially oblong cross-sectional shape. In some embodiments, the suture eyelet 114 may be preloaded with one or more sutures.

In some aspects, the present invention relates to a suture hole located proximal to suture eyelet 114 for receiving at least one suture. In some embodiments, suture hole 128 is a hole passing through body 104 of anchor 102 and allows at least one suture 140 to pass through. In some embodiments, anchor 102 is held firmly against the distal end of driver 130 when suture hole 114 is loaded with suture, and when tension is applied to the suture. In some embodiments, anchor 102 comprises suture hole 128 proximal to suture eyelet 114 to facilitate the receiving, sliding, securing or fixation of pre-loaded and/or knotless suture. In some embodiments, anchor 102 comprises suture hole 128 extending through body 104 creating a lumen wherein suture may pass through. In some embodiments, suture hole 128 traverses the body of anchor 102. Suture hole 128 is configured to receive suture threads and is formed by a lumen passing through the body 104 of anchor 102 from a first lateral opening to an opposing second lateral opening. Suture hole 128 can have any suitable cross-sectional shape, including oblong, circular, elliptical, rectangular, and the like. In some embodiments, suture hole 128 is sized to freely pass a plurality of suture threads as well as any knots formed in the suture threads. In some embodiments, suture hole 128 comprises a substantially oblong cross-sectional shape. In some embodiments, suture hole 128 may be preloaded with one or more sutures. In some embodiments, suture 140 passes through suture hole 128, and when tension is applied to suture 140, anchor 102 is held firmly against the distal end of driver 130. In some embodiments, suture hole 128 is a non-captivating feature that allows the suture to freely move horizontally in the recess to be tensioned or loosened.

In some aspects, the present invention relates to at least one suture recess 118 on anchor 102. Again referring to FIG. 6B, suture recess 118 extends along a portion of body 104 and comprises at least one cut out region in body 104. In some embodiments, suture recess 118 is a relief in body 104 from the proximal end of suture hole 128 to proximal face 124. In one example, anchor 102 has recess 118 on laterally opposed sides aligning with the openings of suture hole 128, and extending to the proximal end of anchor 102. In some embodiments, suture may pass unrestricted from the proximal end of anchor 102, along a first suture recess 118, through suture hole 128 and along a second suture recess 118.

Aspects of the present invention relate to the dimensions for any anchor 102 of the present invention. The body 104 of anchor 102 can have any desired dimensions. In some embodiments, body 104 has a length and a diameter. In various embodiments, anchor body 104 can have a length between about 4 mm to about 50 mm and a diameter between about 2 mm to about 30 mm. For example, body 104 may be 2.5 mm in diameter and 12 mm in length. Body 104 can be described as having an outer diameter or major diameter and an inner diameter or minor diameter, wherein the outer or major diameter refers to a diameter of body 104 including exterior structures (such as threads, ribs, or flaps), and the inner or minor diameter refers to a diameter of body 104 excluding exterior structures. In some embodiments, the size of the exterior structures can be characterized in terms of a difference between the outer/major diameter and the inner/minor diameter. For example, in various embodiments, the outer/major diameter and the inner/minor diameter can differ by between about 0.2 mm to 2 mm, such that the exterior structures extend out from body 104 by a distance between about 0.1 mm to 1 mm.

In some embodiments, suture anchor 102 comprises one or more bone in growth features to enhance anchor pullout strength and biocompatibility. In some embodiments, body 104 of suture anchor 102 comprises at least one outer surface wherein one or more bone in growth features may enhance anchor pullout strength or biocompatibity. Example of bone in growth features include, but are not limited to, laser etching, porous coatings, biocompatible coatings, biopolymer coatings, and the like. Although example bone in growth features are provided, any bone in growth feature may be applied to the at least one outer surface of body 104 as would be known by one of ordinary level of skill in the art.

Aspects of the present invention relate to a mating interface and/or engagement structure for an exemplary driver and anchor according to aspects of the present invention. Anchor 102 may comprise one or more engagement structures compatible with a distal end of driver 130. In some embodiments, driver 130 comprises a distal end opening 142 for receiving at least a portion of proximal end post 126 of anchor 102. For example, as depicted in FIG. 6A, anchor 102 has proximal end post 126 with a post sized to match the distal end opening 142 of driver 130, wherein the engagement between proximal end post 126 and distal end opening 142 may be a friction fit.

Aspects of the invention relate to an anchor having a proximal end post for engaging with a driver, wherein the driver has an opening configured to engage with the post. In some embodiments, anchor 102 has proximal end post 126 for receiving an anchor driver. In some embodiments, proximal end post 126 is a structure, tine, post or protrusion positioned on proximal face 124 to receive the distal end of driver 130. Now referring again to FIG. 6A, in some embodiments, driver 130 may comprise distal end opening 142 configured to engage with proximal end post 126 to form a friction fit wherein the anchor may be inserted and/or rotated into bone. In some embodiments, proximal end post 126 is square in shape. In some embodiments, proximal end post 126 is rectangular in shape. In some embodiments, proximal end post 126 is polygonal in shape. In some embodiments, proximal end post 126 is star shaped. In some embodiments, distal end opening 142 is square in shape. In some embodiments, distal end opening 142 is rectangular in shape. In some embodiments, distal end opening 142 is polygonal in shape. In some embodiments, distal end opening 142 is star shaped. In some embodiments, when driver 130 is fully engaged with anchor 102, distal face 135 of driver 130 contacts proximal face 124 of anchor 102. In some embodiments, distal face 135 of driver 130 prevents proximal end post 126 from extending too far into distal end opening 142.

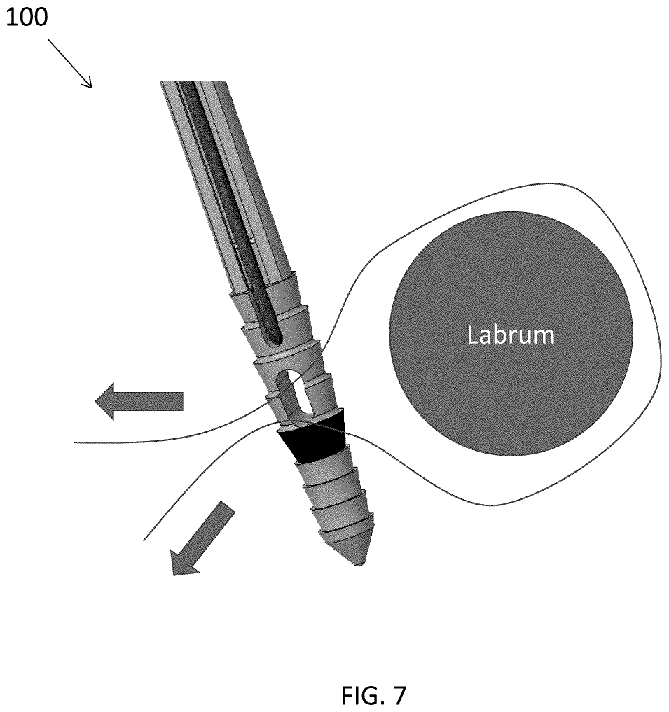

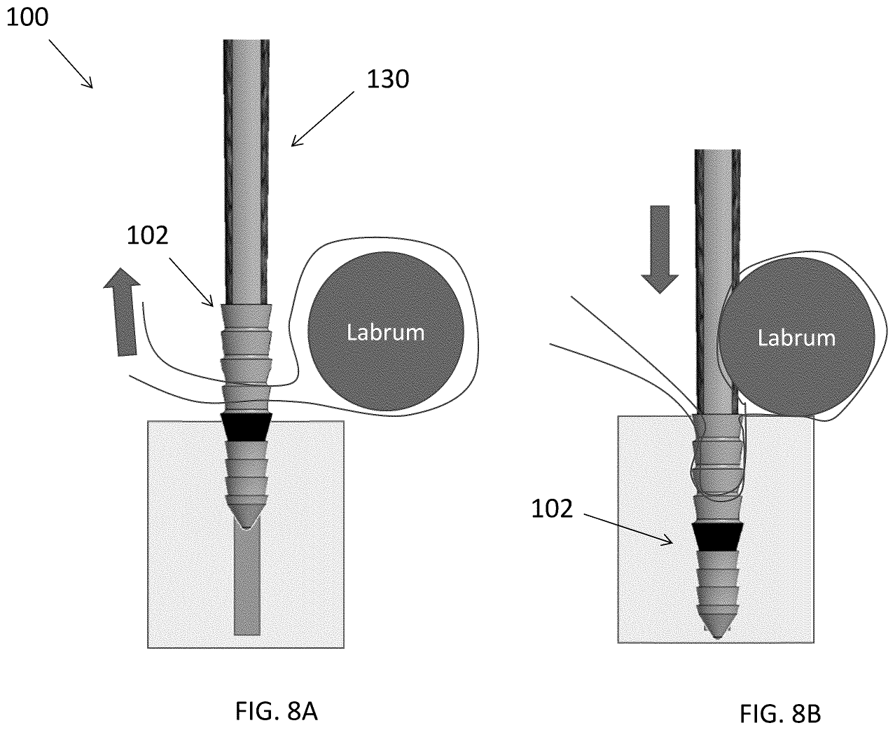

Referring now to FIG. 8A and FIG. 8B, in some embodiments, anchor 102 is impacted to a first position, wherein suture is passed through eyelet 114 of the anchor unobstructed, and the suture is tensioned. Following this, anchor 102 may be impacted to a second or final position, wherein the anchor creates a friction interference with the suture, and locks the suture between the body of the anchor and the surrounding bone, forming a friction lock with the bone wherein the suture is immobilized.

Aspects of the present invention relate to the positioning of anchor 102 in bone or tissue, and visual aids for helping position the anchor correctly. In some embodiments, anchor 102 comprises a laser line 160 positioned on the outer surface of body 104, distal to eyelet 114. In some embodiments, laser line 160 aids in positioning the anchor when driving the anchor into bone and/or tissue. In some embodiments, laser line 160 is a circumferential band of a different color to the rest of body 104, providing a visual aid to the individual positioning the anchor. Laser line 160 is positioned distal on body 104 to eyelet 114 to indicate when a sufficient first position is reached. In some embodiments, when laser line 160 is aligned with the surface of the bone, eyelet 114 allows suture to pass freely through. When the anchor 102 is driven into a second and/or final position, laser line 160 is surrounded by bone, and eyelet 114 does not allow suture to pass freely through.

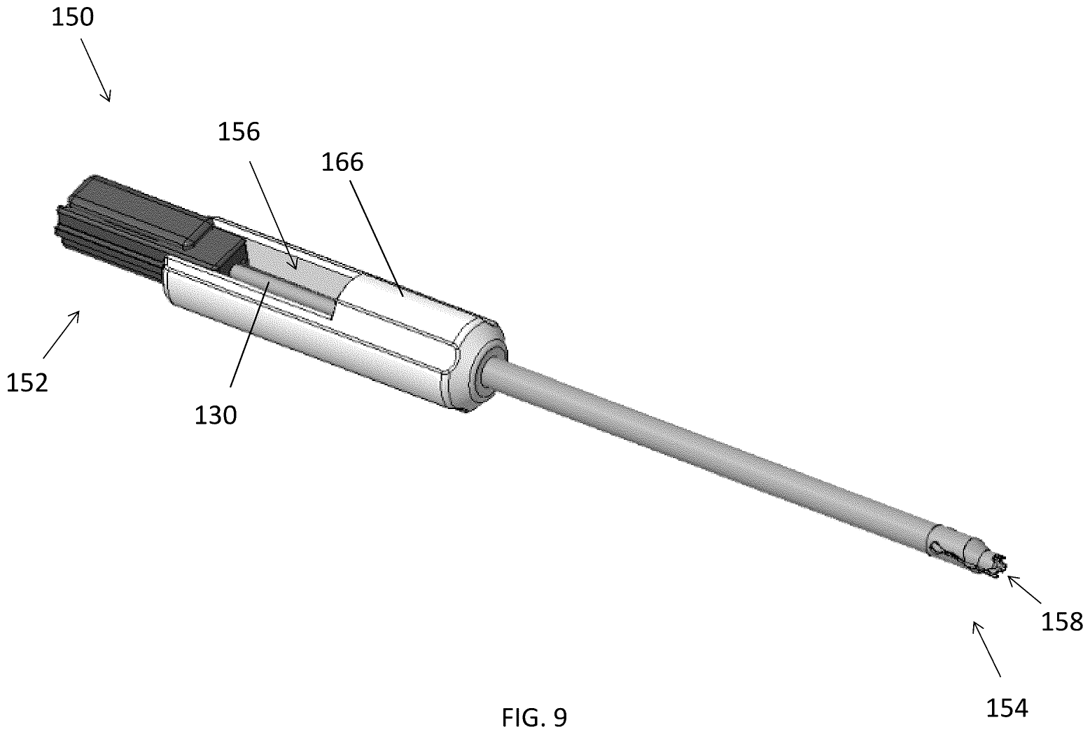

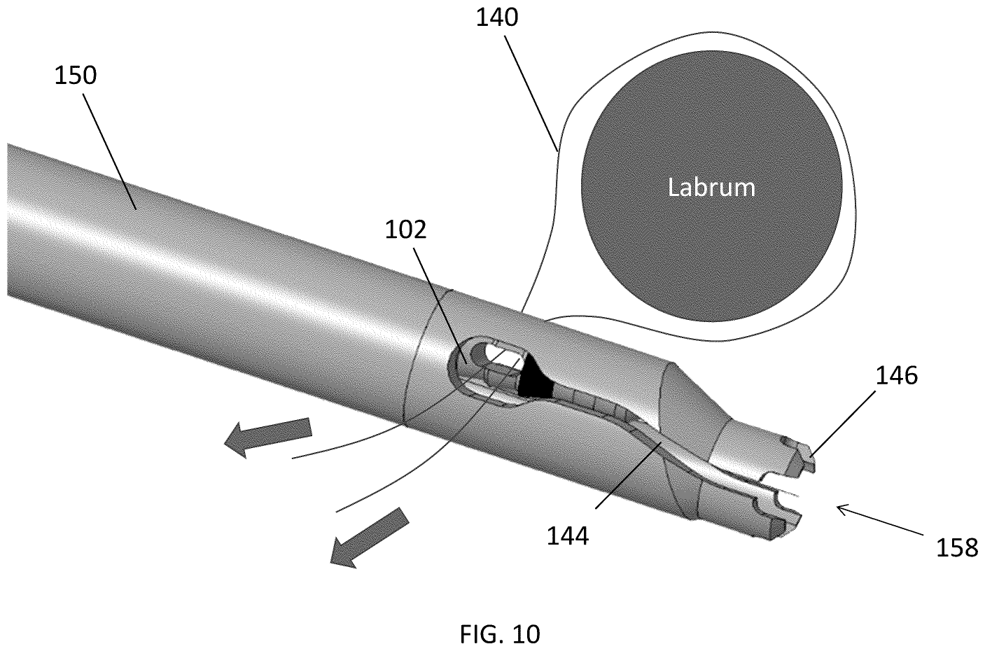

In some aspects, the present invention relates to an anchor system comprising an insertion tool used for inserting an anchor into bone wherein the anchor, driver and drill are situated inside the lumen of the insertion tool. Referring now to FIG. 9, in some embodiments, insertion tool 150 comprises proximal end 152 and distal end 154 and a length therebetween. In some embodiments, insertion tool 150 comprises at least one lumen that runs the length of the tool, the at least one lumen having a proximal end opening 156 and a distal end opening 158. In some embodiments, insertion tool 150 comprises proximal handle 166 used for gripping the tool. Referring now to FIG. 10, in some embodiments, insertion tool 150 has at least one suture guide 144 comprising cut-out regions on laterally opposed sides of the distal end of insertion tool 150, for guiding preloaded suture during the insertion of anchor 102 into bone. In some embodiments, insertion tool 150 comprises one or more distal prongs 146 for stabilizing insertion tool 150 to bone during drilling and the insertion of the anchor into bone.

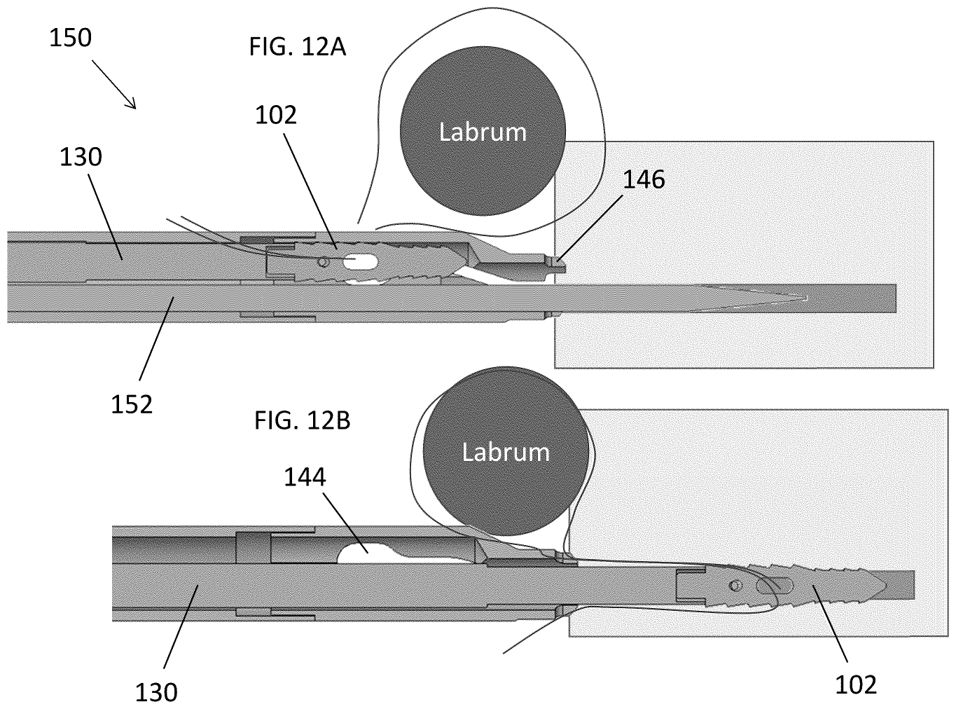

Accordingly, as shown in FIGS. 9-11 and explained in further detail below with reference to FIG. 12A and FIG. 12B, embodiments of the suture anchor system provide superior control and accuracy for surgeons. The suture anchor system according to one embodiment can include an anchor having an elongate cylindrical body extending between a proximal end and a distal end, and an eyelet traversing a portion of the elongate cylindrical body. The drill guide can have an interior chamber in fluid communication with a distal opening. An elongate anchor inserter has a distal surface configured to interface with a proximal surface of the anchor. An elongate drill member has a distal drilling tip. Advantageously, the interior chamber is configured to at least partially house the elongate anchor inserter and the elongate drill member, which simplifies the procedure and improves accuracy.

The distal opening is configured to accept advancement therethrough of the distal drilling tip while the anchor and elongate anchor inserter remain housed in the lumen. A surface of the interior chamber can include a ramp structure that tapers towards the distal opening. The ramp structure can terminate at a distal chamber portion which has a smaller diameter than portions of the interior chamber proximal to the ramp structure. The interior chamber can be configured to arrest movement of the anchor and elongate anchor inserter while the distal drilling tip is advanced through the distal opening. The interior chamber is configured to allow advancement of the anchor and elongate anchor inserter through the distal opening after the elongate drill member is retracted from the distal opening. Advancement of the anchor and elongate anchor inserter through the distal opening can happen in certain embodiments after the elongate drill member is retracted from the distal opening but remains in part of the interior chamber, or in certain embodiments after the elongate drill member is retracted from the interior chamber entirely. The elongate anchor inserter and the elongate drill member are housed in parallel within the interior chamber. The interior chamber can for example have a single lumen configured to house both the elongate anchor inserter and the elongate drill member. Alternatively, a first lumen can be configured to house the elongate anchor inserter and a second lumen can be configured to house the elongate drill member, such that the first and second lumen are in fluid communication with the distal opening.

A suture guide slot is configured in a wall of the interior chamber and terminating distally at the distal opening. The wall includes a first and second portion of the interior chamber, the second portion of the interior chamber configured distal of the first portion of the interior chamber, and the second portion of the interior chamber having a smaller diameter than a first portion of the interior chamber. The suture guide slot can have at least one curved portion. The curved portion can be disposed adjacent to a surface of the interior chamber having the ramp structure that tapers towards the distal opening. The suture guide slot can include at least one straight portion. The suture guide includes a proximal portion that tapers down to a narrower distal portion.

The distal surface of the elongate anchor inserter can have a protrusion configured to mate with the anchor. The proximal surface of the anchor can include a protrusion configured to mate with the elongate anchor inserter. The protrusion can be configured to mate with the elongate anchor inserter is configured to insert into a distal opening of the elongate anchor inserter. The anchor includes a proximal opening traversing the elongate cylindrical body configured proximal of the eyelet. A kit including the suture anchor system can include a suture preloaded within the proximal opening, and the anchor preloaded onto the elongate anchor inserter.

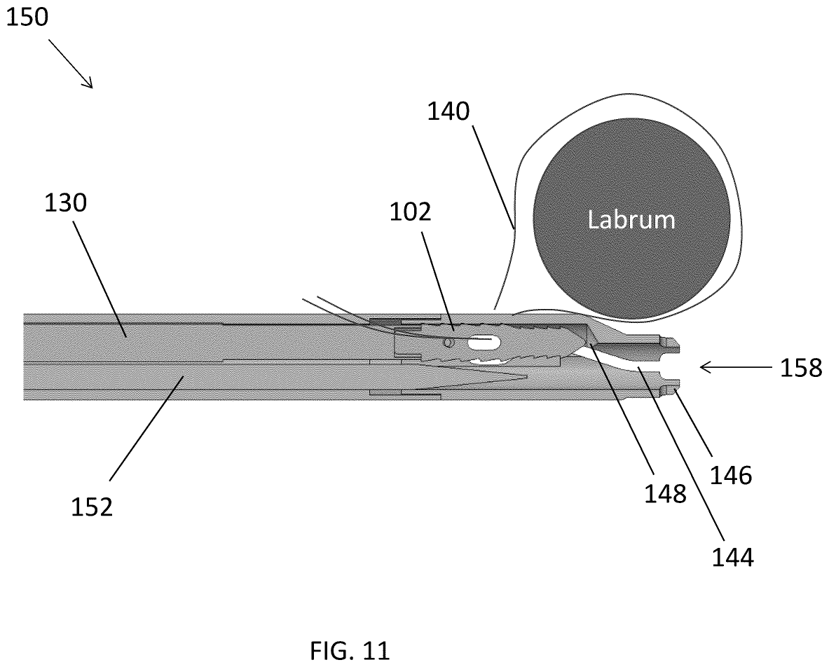

Referring now to FIG. 11, shown is a side view of the distal end of an exemplary insertion tool 150 according to aspects of the present invention. In some embodiments, insertion tool 150 further comprises a driver 130 and a drill 152. In some embodiments, driver 130 and drill 152 may both reside in the lumen of insertion tool 150 simultaneously. In some embodiments, insertion tool 150 further comprises a feed ramp 148.

Referring now to FIG. 12A and FIG. 12B, an exemplary insertion tool 150 comprising an anchor 102, drill 152 and driver 130 is shown. In some embodiments, at least a portion of prongs 146 of insertion tool 150 are inserted into bone. In some embodiments, insertion tool 150 comprises a lumen having a first and second position, wherein anchor 102, driver 130, and/or drill 152 may reside in the first and second positions. In some embodiments, at least one anchor 102 and driver 130 are held in the first position while drill 152 is in the second position. Drill 152 may be used to drill into bone while in the first position via extending drill 152 out of opening 158 and into bone. After the hole is drilled for the anchor, drill 152 is removed from the lumen of insertion tool 150 from the proximal end. In some embodiments, following drilling, driver 130 and anchor 102 are moved from the first position to the second position. In some embodiments, by pushing in a distal direction on driver 130, anchor 102 contacts feed ramp 148, thereby causing driver 130 and anchor 102 to shift from the first position to the second position. In some embodiments, driver 130 may drive anchor 102 into bone by extending driver 130 and anchor 102 out of opening 158 and into the hole drilled by drill 152. In some embodiments, suture 140 resides in suture guide 144 while the hole is drilled, and the anchor is inserted.

In one aspect, the present invention relates to qualities for an driver 130, drill 152 and/or anchor insertion tool 150. In some embodiments, driver 130, drill 152 and/or anchor insertion tool 150 comprises superelastic, pseudoelastic and/or “shape memory” material, such as nitinol, to allow the driver to advance the anchor along a curved path.

In various embodiments, driver 130, drill 152 and/or anchor insertion tool 150 is amenable to any feature or modification to enhance or augment its function. For example, in some embodiments an exterior surface of handle 136 or handle 166 can comprise one or more textured surfaces or ridges configured to enhance a user's grip and to facilitate driving driver 130 or insertion tool 150. In some embodiments, handle 136 or handle 166 can comprise one or more suture holders for temporary cinching or wrapping of suture threads and suture ends. In some embodiments, the proximal end of handle 136 or 166 can comprise a high-density polymer or a metal material configured to withstand repeated strikes from a hammer or mallet with minimal or no deformation.

The suture anchor system comprising any variation of anchors, drivers, drills and insertion tools of the present invention can be made from any suitable material, including but not limited to metals, non-biodegradable polymers, biodegradable polymers, polymer composites, copolymers, and bioceramics. The term biodegradable as used herein is defined to mean materials that degrade in the body and then are either absorbed into or excreted from the body. The term bioceramic as defined herein is defined to mean ceramic and glass materials that are compatible with body tissue and can be biodegradable or non-biodegradable. Contemplated metals include stainless steel, titanium, alloys of nickel and titanium, or other biocompatible metallic materials. The separable components of the various suture anchors described herein may be fabricated from the same material or different materials. For example, in some embodiments anchor body 104 is metal-free and anchor driver 130 comprises metal.

Non-biodegradable polymers include but are not limited to polyethylene, polypropylene, PEEK (polyetheretherketone), PAEK (Polyaryletherketone), bioabsorbables, or other biocompatible non-absorbable polymers. Biodegradable polymers include but are not limited to aliphatic polyesters, polyorthoesters, polyanhydrides, polycarbonates, polyurethanes, polyamides and polyalkylene oxides. In some embodiments, the biodegradable polymers are aliphatic polyester polymers and copolymers, and blends thereof. The aliphatic polyesters are typically synthesized in a ring opening polymerization. Suitable monomers include but are not limited to lactic acid, lactide (including L-, D-, meso and D,L mixtures), glycolic acid, glycolide, epsilon, caprolactone, p-dioxanone (1,4-dioxan-2-one), trimethylene carbonate (1,3-dioxan-2-one), delta-valerolactone, and combinations thereof.

Bioceramics include but are not limited to ceramics comprising mono-, di-, tri-, alpha-tri-, beta-tri-, and tetra-calcium phosphate, hydroxyapatite, calcium sulfates, calcium oxides, calcium carbonates, magnesium calcium phosphates. In addition to bioceramics, bioglasses may also be used. The bioglasses may include phosphate glasses and bioglasses.

Additional contemplated polymers can include poly(amino acids), copoly(ether-esters), polyalkylene oxalates, polyamides, tyrosine derived polycarbonates, poly(iminocarbonates), polyorthoesters, polyoxaesters, polyamidoesters, polyoxaesters containing amine groups, poly(anhydrides), polyphosphazenes, polyurethanes, poly(ether urethanes), poly(ester urethanes), polypropylene fumarate), poly(hydroxyalkanoate), homopolymers and copolymers of lactide (which includes lactic acid, D-,L- and meso lactide); glycolide (including glycolic acid); .epsilon-caprolactone; p-dioxanone (1,4-dioxan-2-one); trimethylene carbonate (1,3-dioxan-2-one); alkyl derivatives of trimethylene carbonate; .delta.-valerolactone; .beta.-butyrolactone; .gamma.-butyrolactone; .epsilon.-decalactone; hydroxybutyrate; hydroxyvalerate; 1,4-dioxepan-2-one (including its dimer 1,5,8,12-tetraoxacyclotetradecane-7,14-dione); 1,5-dioxepan-2-one; 6,6-dimethyl-1,4-dioxan-2-one; 2,5-diketomorpholine; pivalolactone; .alpha.,.alpha.diethylpropiolactone; ethylene carbonate; ethylene oxalate; 3-methyl-1,4-dioxane-2,5-dione; 3,3-diethyl-1,4-dioxan-2,5-dione-; 6,6-dimethyl-dioxepan-2-one; 6,8-dioxabicycloctane-7-one and polymer blends thereof. Additional exemplary polymer or polymer blends include, by non-limiting example, a polydioxanone, a polyhydroxybutyrate-co-hydrox-yvalerate, polyorthocarbonate, a polyaminocarbonate, and a polytrimethylene carbonate.

### Methods of Repairing Soft Tissue to Bone

The present invention also provides methods of repairing soft tissue to bone using the labral suture anchor devices described herein. In various embodiments, the soft tissue can be a tendon or a ligament. In some embodiments, the methods are useful in securing soft tissue to bone, such as in rotator cuff repair.

In some embodiments, the anchor is driven into a preformed hole in the bone, which may be formed using a tool such as an awl, tap, drill, or the like. In some embodiments, the anchor is punched or hammered directly into bone without a preformed hole. In some embodiments, the anchor is configured for push-in insertion. Push-in insertion refers to an insertion method that is not entirely reliant on a screwing-in motion. The anchor can be push-in inserted by hammering or back-and-forth twisting motions, either into a pre-existing hole or directly into native tissue.

In some embodiments, a method comprising an anchor with a preloaded suture through one or more eyelets in the anchor may be passed through soft tissue to secure the soft tissue to the bone. In some embodiments, the anchor may use one or more sutures and/or one or more suture tapes or any combination thereof to secure the soft tissue to bone using any previously disclosed anchors, systems or methods described above.

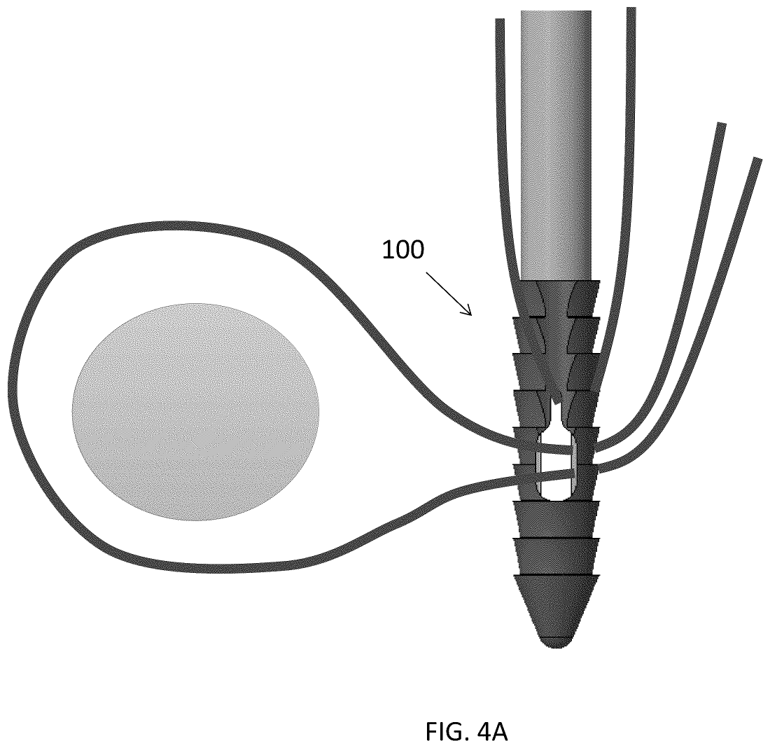

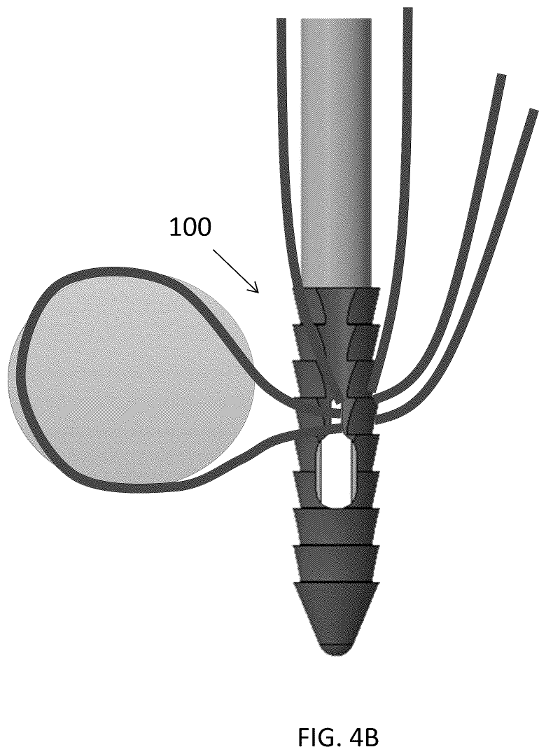

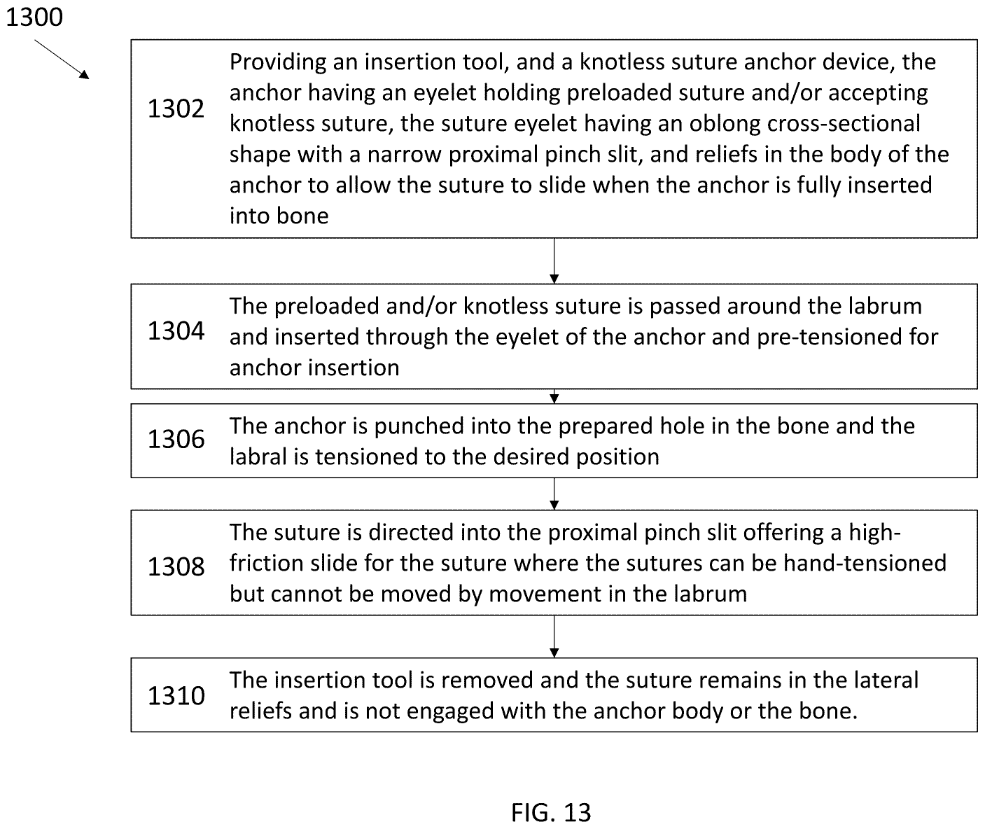

In some aspects, the present invention relates to a method of repairing soft tissue to bone. Referring now to FIG. 13, an exemplary method 1300 of repairing soft tissue to bone is depicted wherein a knotless technique for labral repair allows the surgeon to slowly tension and finetune the position of the labrum without the need for tying a knot. The steps of method 1300 are visually depicted in FIG. 3 through FIG. 5. Method 1300 begins with step 1302, providing an insertion tool, and a labral suture anchor device, the anchor having a longitudinal eyelet holding preloaded suture and/or accepting knotless suture, the suture eyelet having an oblong cross-sectional shape with a narrow proximal pinch slit, and reliefs in the body of the anchor to allow the suture to slide when the anchor is fully inserted into bone (FIGS. 3A and 3B). In step 1304, the preloaded and/or knotless suture is passed around the labrum and inserted through the eyelet of the anchor (FIG. 4A) and pre-tensioned for anchor insertion (FIG. 4B). In step 1306, the anchor is punched into the prepared hole in the bone (FIG. 5A) and the suture is tensioned to the desired position (FIG. 5B). In step 1308, the suture is directed into the pinch slit offering a high-friction slide for the suture wherein the suture may be hand-tensioned but cannot be moved by movement in the labrum. In step 1310, the insertion tool is removed wherein the excess suture remains in the lateral reliefs and not engaged with the anchor ribs or the pinched between the anchor and the bone. In some embodiments, the suture anchor is driven into a preformed hole in the bone, which may be formed using a tool such as an awl, tap, drill, or the like. In some embodiments, the suture anchor is punched or hammered directly into bone without a preformed hole.

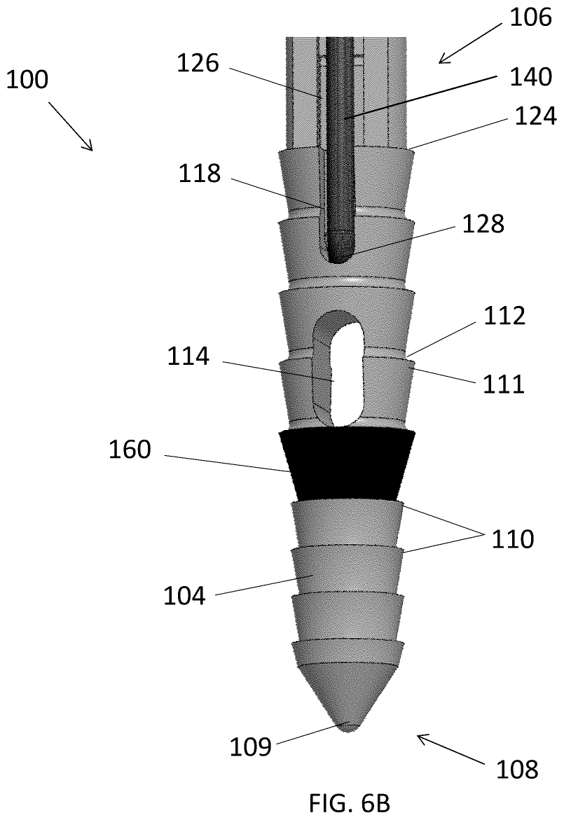

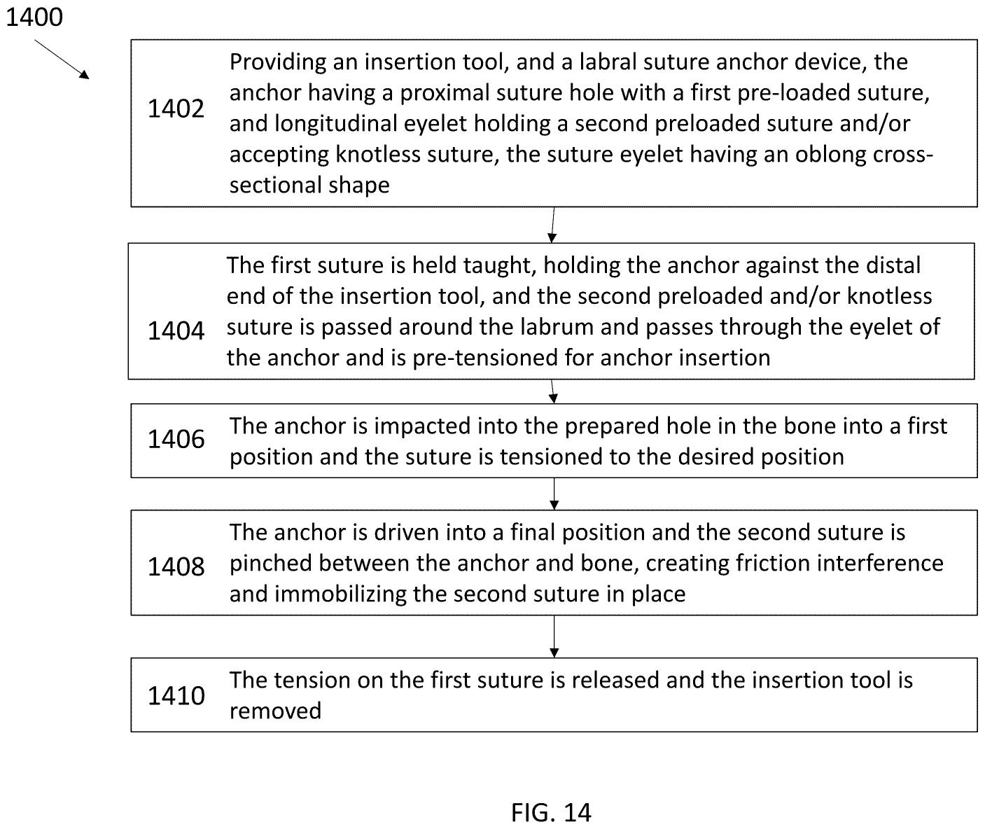

In some aspects, the present invention relates to a method of repairing soft tissue to bone. Referring now to FIG. 14, an exemplary method 1400 of repairing soft tissue to bone is depicted wherein an anchor with at least one suture allow for labral repair. The steps of method 1400 are visually depicted in FIG. 7 through FIG. 8. Method 1400 begins with step 1402, providing an insertion tool, and a labral suture anchor, the anchor having a proximal suture hole with a first pre-loaded suture, and longitudinal eyelet holding a second preloaded suture and/or accepting knotless suture, the suture eyelet having an oblong cross-sectional shape (FIG. 7). In step 1404, the first suture is held taught, holding the anchor against the distal end of the insertion tool, and the second preloaded and/or knotless suture is passed around the labrum and passes through the eyelet of the anchor and is pre-tensioned for anchor insertion (FIG. 7). In step 1406, the anchor is impacted into the prepared hole in the bone to a first position and the suture is tensioned to the desired position (FIG. 8A). In step 1408, the anchor is driven into a final position and the second suture is pinched between the anchor and bone, creating friction interference and immobilizing the second suture in place (FIG. 8B). In step 1410, the tension on the first suture is released and the insertion tool is removed. In some embodiments, the first suture is also passed around the labrum adding an additional restraint to the labrum. In some embodiments, the suture anchor is driven into a preformed hole in the bone, which may be formed using a tool such as an awl, tap, drill, or the like. In some embodiments, the suture anchor is punched or hammered directly into bone without a preformed hole.

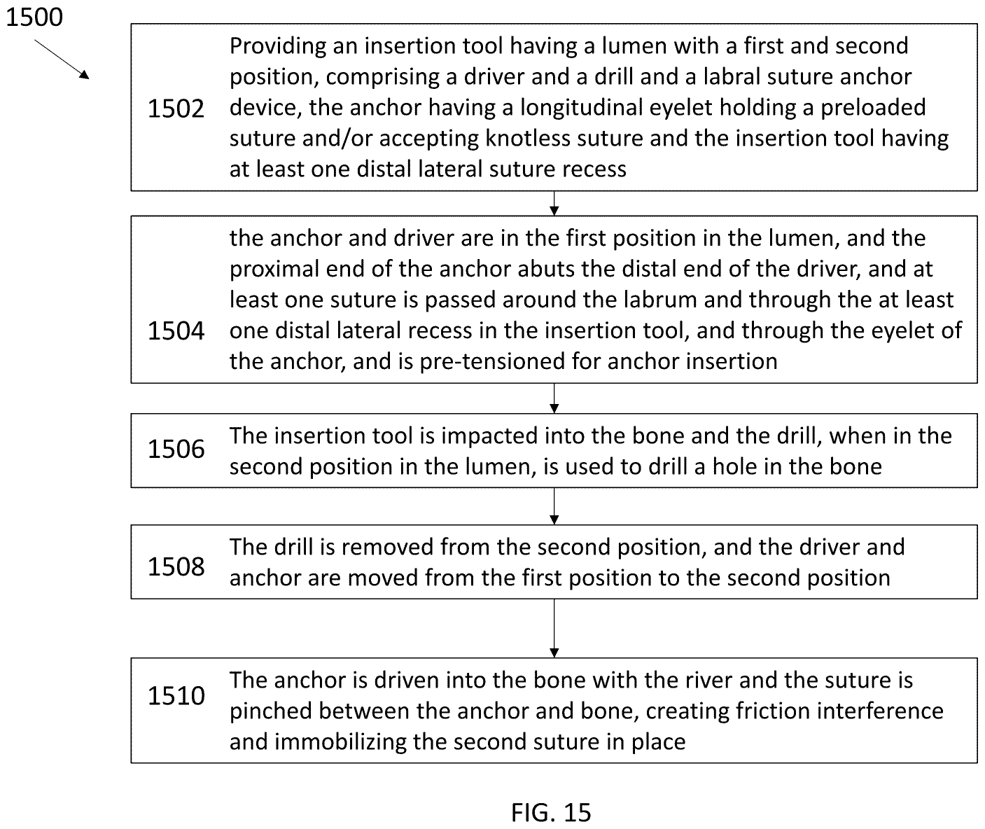

In some aspects, the present invention relates to a method of repairing soft tissue to bone. Referring now to FIG. 15, an exemplary method 1500 of repairing soft tissue to bone is depicted wherein an insertion tool has both a drill and a driver, and allows for a hole to be drilled in the bone, and the anchor to be inserted without removing the insertion tool from the site. The steps of method 1500 are visually depicted in FIG. 11 and FIG. 12A and FIG. 12B. Method 1500 begins with step 1502, providing an insertion tool having a lumen with a first and second position, comprising a driver and a drill and a labral suture anchor, the anchor having a longitudinal eyelet holding a preloaded suture and/or accepting knotless suture and the insertion tool having at least one distal lateral suture recess (FIG. 11). In step 1504, the anchor and driver are in the first position in the lumen, and the proximal end of the anchor abuts the distal end of the driver, and at least one suture is passed around the labrum and through the at least one distal lateral recess in the insertion tool, and through the eyelet of the anchor, and is pre-tensioned for anchor insertion (FIG. 11). In step 1506, the insertion tool is impacted into the bone and the drill, when in the second position in the lumen, is used to drill a hole in the bone (FIG. 12A). In step 1508, the drill is removed from the second position, and the driver and anchor are moved from the first position to the second position (FIG. 12B). In step 1510, the anchor is driven into the bone with the river and the suture is pinched between the anchor and bone, creating friction interference and immobilizing the second suture in place (FIG. 12B).

In some embodiments, the methods of the present invention can be repeated for one or more anchor devices for fixation of soft tissue at one or more locations along the bone. The number and spacing of the anchors may be varied depending on the particular application and extent of an injury. In some embodiments, one or more of the anchor devices can be implanted into bone to form a medial row of anchors, a lateral row of anchors, or both as would be understood by persons having skill in the art that are versed in rotator cuff repair. In one example, the medial row of anchors and the lateral row of anchors are positioned beneath soft tissue (such as a tendon) such that the soft tissue completely covers each of the anchors.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A suture anchor system comprising:
an anchor having an elongate cylindrical body extending between a proximal end and a distal end, and an eyelet traversing a portion of the elongate cylindrical body;
a drill guide having an interior chamber in fluid communication with a distal opening;
an elongate anchor inserter having a distal surface configured to interface with a proximal surface of the anchor; and
an elongate drill member having a distal drilling tip;
wherein the interior chamber is configured to at least partially house the elongate anchor inserter and the elongate drill member.

2. The suture anchor system of claim 1, wherein the distal opening is configured to accept advancement therethrough of the distal drilling tip while the anchor and elongate anchor inserter remain housed in the lumen.

3. The suture anchor system of claim 2, wherein a surface of the interior chamber comprises a ramp structure that tapers towards the distal opening.

4. The suture anchor system of claim 3, wherein the ramp structure terminates at a distal chamber portion which has a smaller diameter than portions of the interior chamber proximal to the ramp structure.

5. The suture anchor system of claim 2, wherein the interior chamber is configured to arrest movement of the anchor and elongate anchor inserter while the distal drilling tip is advanced through the distal opening.

6. The suture anchor system of claim 2, wherein the interior chamber is configured to allow advancement of the anchor and elongate anchor inserter through the distal opening after the elongate drill member is retracted from the distal opening.

7. The suture anchor system of claim 2, wherein the elongate anchor inserter and the elongate drill member are housed in parallel within the interior chamber.

8. The suture anchor system of claim 1, wherein the interior chamber comprises a single lumen configured to house both the elongate anchor inserter and the elongate drill member.

9. The suture anchor system of claim 1, wherein the interior chamber comprises a first lumen configured to house the elongate anchor inserter and a second lumen configured to house the elongate drill member, wherein the first and second lumen are in fluid communication with the distal opening.

10. The suture anchor system of claim 1, comprising a suture guide slot configured in a wall of the interior chamber and terminating distally at the distal opening.

11. The suture anchor system of claim 10, wherein the wall comprises a first and second portion of the interior chamber, the second portion of the interior chamber configured distal of the first portion of the interior chamber, and the second portion of the interior chamber having a smaller diameter than a first portion of the interior chamber.

12. The suture anchor system of claim 10, wherein the suture guide slot comprises at least one curved portion.

13. The suture anchor system of claim 12, wherein the curved portion is disposed adjacent to a surface of the interior chamber comprising a ramp structure that tapers towards the distal opening.

14. The suture anchor system of claim 10, wherein the suture guide slot comprises at least one straight portion.

15. The suture anchor system of claim 10, wherein the suture guide comprises a proximal portion that tapers down to a narrower distal portion.

16. The suture anchor system of claim 1, wherein the distal surface of the elongate anchor inserter comprises a protrusion configured to mate with the anchor.

17. The suture anchor system of claim 1, wherein the proximal surface of the anchor comprises a protrusion configured to mate with the elongate anchor inserter.

18. The suture anchor system of claim 17, wherein the protrusion configured to mate with the elongate anchor inserter is configured to insert into a distal opening of the elongate anchor inserter.

19. The suture anchor system of claim 1, wherein the anchor comprises a proximal opening traversing the elongate cylindrical body configured proximal of the eyelet.

20. A kit comprising:
the suture anchor system of claim 19;
a suture preloaded within the proximal opening; and
the anchor preloaded onto the elongate anchor inserter.

21-73. (canceled)
